(19) United States
(12) Patent Application Publication
(10) Pub. No.: US 2005/0096349 A1
(43) Pub. Date: May 5, 2005

(54) Methods to prevent or ameliorate medication-, procedure-or stress-induced cognitive and speech dysfunction and methods to optimize cognitive and speech functioning

(75) Inventors: Nathan Andrew Shapira, Gainesville, FL (US); Giselle D. Mann, Gainesville, FL (US); April M. Annis, Gainesville, FL (US); Toby Doris Goldsmith, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida, Gainesville, FL (US)

(21) Appl. No.: 10/700,156

(22) Filed: Nov. 3, 2003

(51) Int. Cl. A61K 31/445; A61K 31/137

(52) U.S. Cl. 514/317; 514/649

(57) ABSTRACT

Many medications, herbal remedies and procedures have side affects that contribute to cognitive dysfunction. Because of these side effects patients may choose to discontinue medication or treatment (such as ECT) which has a detrimental effect on treatment. Currently, there are no medications approved for helping to alleviate these symptoms. The invention describes a novel treatment for individuals who experience various side effects of cognitive dysfunction. The invention additionally describes how these medications may enhance cognitive function in individuals with normal cognitive functioning who might benefit from this type of enhancement. The subject invention further provides materials and methods for the treatment, prevention, or ameliorate medication-induced cognitive dysfunction comprising the administration of medications or compositions comprising at least one selective norepinephrine reuptake inhibitors (SNR1) and/or buproprion.

### BACKGROUND OF INVENTION

Numerous medications, (prescription and nonprescription) have adverse side effects that contribute to cognitive and speech dysfunction. These side effects include: mental slowness or dulling, speech abnormalities, confusion, memory loss, abnormal thinking, and feeling strange. These cognitive side effects may lead to medication noncompliance as well as morbidity and mortality. Medication types, such as barbituates and selective serotonin reuptake inhibitors, can have high rates of reported cognitive dysfunction. (Petty F, Davis L L, Kabel D, Kramer G L, 1996).

Examples of medication-induced cognitive dysfunction can be found on the PDR Electronic Library™ Online:© 2002-2003. Anti-seizure medications such as zonisamide (Zonegran®) and topiramate (Topamax®) have side effects including: difficulty concentrating, difficulty with memory, mental slowing, speech abnormalities, difficulty with verbal expression, difficulty with concentration or attention, confusion, language problems and decreased cognitive performance. Sleep medication such as zolpidem (Ambien®) have side effects including: drugged feeling, confusion, decreased cognition, detachment, difficulty concentrating, speech disorder, dysarthria, abnormal thinking, feeling strange, intoxicated feeling and amnesia (memory problems). Anti-Inflammatory medications such as Muromonab-CD3 (Orthoclone OKT3 Sterile Solution®), Naproxen Sodium (Anaprox Tablets®) and Naproxen Sodium (Naproxen Sodium®) have side effects including: confusion, impaired cognition, inability to concentrate, amnesia, confusion, and cognitive dysfunction. Psoriasis/Arthritis medications such as Metotrexate Sodium (Methotrexate Sodium Tablets®) and Methotrexate Tablets (Trexall Tablets®) have side effects including: malaise, cognitive dysfunction and speech impairment (dysarthria). Antidepressant medications such as: Paroxetine (Paxil®) and Fluoxetine (Prozac®) have side effects including drugged feeling, confusion, depersonalization, amnesia, dysarthria and abnormal thinking. Medication for severe diarrhea, such as Alosetron Hydrochloride (Latronex®) have side effects including: memory effects, cognitive function disorders and confusion. Opiate medications such as Tramadol Hydrochloride Tablets (Ultram Tablets®) have side effects including: confusion and cognitive dysfunction. Anti-anxiety medications such as Alprazolam Tablets®, UPS (Xanax®) and Diazepam (Valium®) have side effects including: memory impairment, cognitive disorder, confused state, and dysarthria. Herbal Medicine can also have cognitive dysfunction side effects.

Since there is not an approved medication by the FDA to prevent medication induced cognitive dysfunction, many individuals self-medicate. The most common drug used to gain focus is caffeine (Smit H J, Rogers P J, 2000). However, caffeine has side effects that impact cognitive function once individuals are deprived from the substance (Kourtidou-Papadeli C, Papadelis C et al., 2002). Side effects can develop as soon as the first missed cup of coffee, 100 mg of caffeine, or even with a missed dose of 50 mg or less, the amount in a typical serving of tea or a caffeinated cola (Lane J, 1997). Symptoms of withdrawal can last up to one week if there is no caffeine intake (Lane J, 1997). Many individuals experience symptoms including: headaches, irritability, blurred vision, fatigue, tiredness, difficulty with thinking, and working and feeling generally unwell (Lane J, 1997). Also, over time, individuals adjust to the intake of caffeine, so that more caffeine is needed to experience the effects. This built up tolerance, means that habitual caffeine drinkers need to increase their dose of caffeine to achieve optimal performance. Thus, the optimal dose of caffeine to achieve an increase in cognitive performance depends on the individual, and can change over time (Lieberman H R, 2001).

Another over-the-counter drug commonly used to attempt to improve cognitive functioning is Ephedrine (Schaeberg B T, Crockett S et al., 2003). Ephedrine is an agent that stimulates the Central Nervous System (CNS), and Cardiovascular System (CS) (Lane J, 1997). Individuals can experience adverse reactions such as palipatations, stress, headache, and insomnia. Some of the severe reactions to ephedrine include stroke, heart attack, cardiac arrhythmias, seizures and psychotic disorders (Van der Hooft C S, Stricker B H, 2002). Products containing ephedrine are not to be used by people with heart disease, hypertension, diabetes, thyroid disease, enlarged prostate, anxiety and restlessness, glaucoma, people taking monamine oxidase inhibitors or woman who are pregnant or lactating (Van der Hooft C S, Stricker B H, 2002). Due to the significant side effect profile of ephedrine, the use of this herbal medication is not ideal.

### BRIEF SUMMARY OF THE INVENTION

Many medications, herbal remedies and procedures have side affects that contribute to cognitive dysfunction. Because of these side effects patients may choose to discontinue medication or treatment (such as ECT) which has a detrimental effect on treatment. Currently, there are no medications approved for helping to alleviate these symptoms. The invention describes a novel treatment for individuals who experience various side effects of cognitive dysfunction. The invention additionally describes how these medications may enhance cognitive function in individuals with normal cognitive functioning who might benefit from this type of enhancement. The subject invention further provides materials and methods for the treatment, prevention, or ameliorate medication-induced cognitive dysfunction comprising the administration of medications or compositions comprising one or more selective norepinephrine reuptake inhibitors (SNR1) and/or buproprion.

### DETAILED DISCLOSURE OF THE INVENTION

The subject invention provides materials and methods for the treatment, prevention, or ameliorate medication-induced cognitive dysfunction comprising the administration of medications or compositions comprising buproprion and/or one or more selective norepinephrine reuptake inhibitor (SNR1). Non-limiting examples of SNR1 include reboxetine, atomoxetine, oxaprotiline, desiparamine, nisoxetine, ludiomil, and fezolamine. Compositions comprising one or more SNR1 can be co-administered with the affecting medication. Likewise, compositions comprising buproprion and, optionally, one or more SNR1 can be co-administered with an affecting composition. Reboxetine is a clinically effective antidepressant drug that does not tend to cause cognitive dysfunction, unlike selective serotonin reuptake inhibitors (Michelson D, Adler L et al., 2003). Another one of these types of medications is atomoxetine (Strattera®) which is FDA approved for Attention Deficit/Hyperactivity Disorder (ADHD). Strattera is also a clinically effective and safe treatment for ADHD (Pliszka SR). In various aspects of the invention, the compositions used in this method of the subject invention can exclude polypeptides and/or individual amino acids.

Non-limiting examples of medications that can induce cognitive dysfunction include: anti-seizure medications such as zonisamide (Zonegran®) and topiramate (Topamax®) [having side effects including: difficulty concentrating, difficulty with memory, mental slowing, speech abnormalities, difficulty with verbal expression, difficulty with concentration or attention, confusion, language problems and decreased cognitive performance]; sleep medications (such as zolpidem (Ambien®) having side effects including: drugged feeling, confusion, decreased cognition, detachment, difficulty concentrating, speech disorder, dysarthria, abnormal thinking, feeling strange, intoxicated feeling and amnesia (memory problems)); anti-inflammatory medications (such as Muromonab-CD3 (Orthoclone OKT3 Sterile SolutionE), Naproxen Sodium (Anaprox Tablets®) and Naproxen Sodium (Naproxen Sodium®) having side effects including: confusion, impaired cognition, inability to concentrate, amnesia, confusion, and cognitive dysfunction); psoriasis/arthritis medications (such as Metotrexate Sodium (Methotrexate Sodium Tablets®) and Methotrexate Tablets (Trexall Tablets®) having side effects including: malaise, cognitive dysfunction and speech impairment (dysarthria)); anti-depressant medications (such as: Paroxetine (Paxil®) and Fluoxetine (Prozac®) having side effects including drugged feeling, confusion, depersonalization, amnesia, dysarthria and abnormal thinking); medications for severe diarrhea (such as Alosetron Hydrochloride (Latronex®) having side effects including: memory effects, cognitive function disorders and confusion); opiate-based medications such as Tramadol Hydrochloride Tablets (Ultram Tablets®) having side effects including: confusion and cognitive dysfunction); anti-anxiety medications (such as Alprazolam Tablets®, UPS (Xanax®) and Diazepam (Valium®) have side effects including: memory impairment, cognitive disorder, confused state, and dysarthria); and herbal medicines that can also have cognitive dysfunction side effects.

In addition to medication-induced cognitive dysfunction, medical procedures can also be associated with cognitive dysfunction with many individuals being at risk of perioperative cognitive dysfunction (Hirsch C H, 1995). In particular neurocognitive changes have been noted following orthopedic interventions, patients with incomplete or heavy pain control (Duggleby W, Lander J, 1994), post coronary artery bypass graft (Haddock CK, Poston WS et al., 2003), following craniectomy (Ellis K, Speed J et al., 1998), carotid endarterectomy procedures (Heyer E J, Sharma R et al., 2000), and electroconvulsive therapy (ECT) (Neylan T C, Canick J D, Hall S E et. al., 2001). Older patients are at particular risk of perioperative morbidity due to the limited flexibility and reserve of their body systems. Thus, the subject invention also provides methods of: 1) reducing the incidence of; 2) treating; 3) preventing; or 4) ameliorating cognitive dysfunction that is associated with, or arises from, medical procedures such as, but not limited to, surgical interventions (procedures), incomplete or heavy pain control, or electroconvulsive therapy comprising the administration of compositions comprising buproprion and/or one or more SNR1 to an individual. Non-limiting examples of SNR1 include reboxetine, atomoxetine, oxaprotiline, desiparamine, nisoxetine, ludiomil, and fezolamine. In certain aspects of the subject invention, compositions comprising buproprion are administered to the patient. In other aspects of the invention, buproprion and one or more SNR1 are administered to the patient. Yet other aspects of the invention provide for the administration of compositions comprising one or more SNR1 to the patient. Additionally, compositions comprising buproprion and/or SNR1 can be administered before, during, and/or after a particular medical procedure. In various aspects of the invention, the compositions used in this method of the subject invention can exclude polypeptides and/or individual amino acids.

Stressful situations are well known to evoke subtle psychophysical changes in both speech and language performance, even in individuals who usually function in the normal cognitive range. Under certain stressful circumstances persons may experience changes in voice intensity and quality, reductions in speech fluency, difficulty word finding, increased mental slowness, verbal confusion, over use of filled pauses (Phillips G M, Sokoloff K A, 1979). In addition, the use of recreational substances (drugs) to ameliorate these responses often results in associated negative side effects. Thus, the subject invention provides methods of methods of: 1) reducing the incidence of; 2) treating; 3) preventing; or 4) ameliorating cognitive dysfunction that is associated with, or arises from, a stressful situation comprising the administration of buproprion and/or one or more SNR1 to the individual. Compositions comprising buproprion and/or one or more SNR1 can be administered before the stressful situation arises or during the course of the stressful situation. In various aspects of the invention, the compositions used in this method of the subject invention can exclude polypeptides and/or individual amino acids.

There is also a need for a method to optimize cognitive function for individuals who test in the normal cognitive range, but under various circumstances, could benefit from optimized cognitive function. Examples of such a scenario include: individuals taking exams, servicemen and officers in the Armed Services during exercises or armed conflict, students, athletes during sporting events, and individuals in various work-settings. Compositions comprising buproprion and/or one or more SNR1 can be administered to these individuals as needed, before, or during activities that require optimized cognitive function. In various aspects of the invention, the compositions used in this method of the subject invention can exclude polypeptides and/or individual amino acids.

For the purposes of this invention, cognitive dysfunction is defined as: mental slowness or dulling, speech abnormalities, difficulty in word finding, confusion, memory loss, abnormal thinking, feeling strange or intoxicated, difficulty with memory, difficulty with verbal expression, difficulty with concentration or attention, language problems, decreased cognitive performance, drugged feeling, decreased or impaired cognition, detachment, speech disorder, dysarthria, or amnesia (memory problems).

Compositions comprising buproprion and/or one or more SNR1 can be administered at dosages that range from 1 mg to 1000 mg per day and by various routes of administration known to those skilled in the art. Other aspects of the invention provide fixed dosages of buproprion and/or one or more SNR1 that range from 5-500 mg per day, 8-100 mg per day, 50-500 mg per day. Yet other embodiments provide for fixed dosages of SNR1 and/or buproprion that range from 2.5-50 mg/day. Additional dosages of SNR1 and buproprion suitable for use in the subject invention can obtained from readily obtainable sources, such as the Physicians Desk Reference or by assessing a patient at various dosages of SNR1 and/or buproprion.

Still other embodiments of the subject invention vary the dosage of the buproprion and/or SNR1 containing compositions daily. For example, a certain dose of a SNR1 composition (optionally containing buproprion in addition to one or more SNR1) can be administered on a first day followed by a higher or lower dose of a SNR1 composition comprising one or more SNR1 and, optionally buproprion, on a subsequent day. The subsequent day can be the next day or, alternatively, the higher or lower dose of compositions comprising one or more SNR1 and, optionally, buproprion, can be administered one or more days after the administration of the first dose. The desired dosage of buproprion and/or one or more SNR1 can be administered as a single dose or as multiple doses. Compositions can be administered via injection, orally, via suppository, topically, or parenterally.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### EXAMPLE 1

Patient is a 52-year-old female with compulsive shopping (impulse control disorder, not otherwise specified), type II diabetes, and obesity. She has no other psychiatric disorders including mood or anxiety disorders. Patient has experienced decreased urges and improved control over her shopping behaviors on topiramate, particularly as the dose had been slowly increased. However, when topiramate was increased to 75 mg po qd, patient began to have word finding difficulty which persisted. She described that it interfered with her work and that she would be unable to increase topiramate higher than this dose.

Atomoxetine was initiated at 25 mg po qd. Patient described mild improvement in word finding over the next month. Atomoxetine dosage was increased to 40 mg po qd and patient described that over the following month she had noticeable improvement in her word finding and could tolerate a topiramate dose of 100 mg without difficulty. Patient tolerated a dose of 100 mg of topiramate for 2 months while on 40 mg po qd of atomoxetine. Due to improved control over shopping behavior with higher dosages and decreased weight at higher dosages of topiramate, she agreed to continue slowly increasing her topiramate dosage higher while continuing to take atomoxetine 40 mg po qd.

### EXAMPLE 2

Patient is a 54-year-old male with panic disorder and recurrent major depression with a history of taking various antidepressants with poor efficacy. Patient was on escitalopram 20 mg po qd and alprazolam 1 mg up to 4×/daily. He describes feeling dull and having difficulty concentrating which is causing problems at work and even in day-to-day activities (e.g., reading the newspaper). Atomoxetine was initiated at 25 mg po qd and patient describes that within a few days he had improved concentration and improved ability to work. However, when he raised the atomoxetine dose to 40 mg po qd he describes that he did not have the same improvement as at 25 mg and he felt dizzy and “spacy”. By alternating 25 mg and 40 mg po qd every other day he achieved the appropriate balance of improved concentration without significant side effects. His anxiety continued to be high but he was now able to take an effective dose of alpraxolam (2-4 mg/day) without difficulties in concentration/cognitive functioning

### EXAMPLE 3

Patient is a 52 year old female with major depression, panic, generalized anxiety disorder, chronic headaches and fibromyalgia. She is on escitalopram 20 mg po qd, olanzapine 7.5 mg po qhs, clonazepam 0.5 mg po bid, and fiorinal prn. Patient described feeling “cloudy” and “disoriented” and feels that this may relate to her medications such as olanzapine, clonazepam, and fiorinal; however, she indicates that she cannot function without these medications. Atomoxetine was initiated at 18 mg po qd and increased to 25 mg po qd after 8 days. Patient related improved concentration, attention, and “focus” on atomoxetine, that it was a stable effect for 2 months. She has been able to tolerate her other medications without difficulty in terms of cognitive functioning.

### REFERENCES

(1) Bymaster F P, Katner J S, Nelson D L, Henrick-Luecke S K, Threlkald P G, Heilgenstein J H, Morin S M, Gehlert D R, Perry, K W. Atomoxetine increases extracellualr levels of norepiniephrine and dopamine in prefrontal cortex of a rat: a potential mechanism for efficacy in attention deficit/hyperactivity disorder. Neuropharmacology 2002; 27(5): 699-711.
(2) Duggleby W, Lander J. Cognitive status and postoperative pain: older adults. J Pain Symptom Management. 1994 January; 9(1): 19-27.
(3) Ellis K, Speed J, Balbierz J M. Post-craniectomy intracranial hypotension: potential impact on rehabilitation. Brain Inj 1998 October; 12(10): 895-9.
(4) Haddock C K, Poston W S, Taylor J E. Neurocognitive sequelae following coronary bypass graft. A research agenda for behavioral scientists. Behav Modif 2003 January; 27(1): 68-82.
(5) Heyer E J, Sharma R, Rampersad A, Winfree C J, Mack W J, Soloman R A, Todd G J, McCormick P C, McMurtry J G, Quest D O, Stem Y, Lazar R M, Connolly E S. A controlled prospective study of neuropsychological dysfunction following carotid endarterectomy. Arch Neurol. 2000 Feb: 59(2): 217-222.
(6) Hirsch C H. When your patient needs surgery: how planning can avoid complications. Geriatrics 1995 February; 50(2) 39-44.
(7) Kourtidou-Papadeli C, Papadelis C, Louizos A L, guiba-Tziampiri O. Maximum cognitive performance and physiological time trend measurements after caffeine intake. Brain Cogn Brain res. 2002; 13(3): 407-15.
(8) Lane J. Effect of brief caffeinated-Beverage deprivation on mood, symptoms, and psychomotor performance. Phar Bio and Beh, 1997: 58(1): 203-208.
(9) Lieberman H R. The effects of ginseng, ephedrine, and caffeine on cognitive performance, mood, and energy. Nutr. Rev. 2001; 59(4): 91-102.
(10) Michelson D, Adler L, Spencer T, Reimherr F W, West S A, Allen A J, Kelsey D, Wemicke J, Dietrick A, Milton D. Atomoxetine in adults with ADHD: two randomized placebo controlled studies. Biol Psychiatry. 2003; 112-20.
(11) Neylan T C, Canick J D, Hall S E, Reus V I, Sapolsky R M, Wolkowitz O M. Cortisol levels predict cognitive impairment induced by electroconvulsive therapy. Biol Psychiatry 2001 Sep. 1; 50(5):331-6
(12) Petty F, Davis L L, Kabel D, Kramer G L. Serotonin dysfunction disorders: a behavioral neurochemistry perspective. J Clin psychiatry 1996; 57 Supp 8: 11-6.
(13) Phillips G M, Sokoloff K A. An end to anxiety treating speech problems with rhetoritherapy. J Commun Disord. 1979 September; 12(5): 385-97.
(14) Pliszka S R, Non-stimulant treatment of attention-deficit/hyperactivity disorder. CNS Spectr. 2003 April; 8 (4): 253-8.
(15) Schaeberg B T, Crockett S, Bedir E, Khan I A. The role of chemical fingerprinting: application to ephedra: phytochemistry. 2003; 62(6): 911-8.
(16) Smit H J, Rogers P J. Effects of low doses of caffeine on cognitive performance, mood and thirst in low and higher caffeine consumers. Psychopharmacology. 2000; 152(2): 167-73.
(17) Van her Hooft C s, Stricker B H. Ephedrine and ephedra in weight loss products and other preparations. Ned Tijdschr Geneeskd; 2002 Jul. 13; 146(28): 13335-6.

1. A method for the treatment, prevention, or ameliorate of medication-induced cognitive dysfunction comprising the administration of medications or compositions comprising one or more selective norepinephrine reuptake inhibitors (SNR1) or buproprion to an individual.

2. The method according to claim 1, wherein said one or more SNR1 are selected from the group consisting of reboxetine, atomoxetine, oxaprotiline, desiparamine, nisoxetine, ludiomil, and fezolamine.

3. The method according to claim 1, wherein said SNR1 containing compositions are co-administered with an affecting medication.

4. A method for the treatment, prevention, or amelioration of perioperative cognitive dysfunction comprising the administration of medications or compositions comprising buproprion or one or more selective norepinephrine reuptake inhibitors (SNR1) before, during, or after a medical procedure to an individual.

5. The method according to claim 4, wherein said perioperative dysfunction is caused by orthopedic interventions, patients with incomplete or heavy pain control, post coronary artery bypass graft, following craniectomy, carotid endarterectomy procedures, or electroconvulsive therapy (ECT).

6. The method according to claim 5, wherein said one or more SNR1 are selected from the group consisting of reboxetine, atomoxetine, oxaprotiline, desiparamine, nisoxetine, ludiomil, and fezolamine.

7. The method according to claim 4, wherein said one or more SNR1 are selected from the group consisting of reboxetine, atomoxetine, oxaprotiline, desiparamine, nisoxetine, ludiomil, and fezolamine.

8. A method of treating or ameliorating cognitive dysfunction that is associated with, or arises from, a stressful situation comprising the administration of a composition comprising buproprion or one or more SNR1 before or during the stressful situation to an individual.

9. The method according to claim 8, wherein said one or more SNR1 are selected from the group consisting of reboxetine, atomoxetine, oxaprotiline, desiparamine, nisoxetine, ludiomil, and fezolamine.

10. A method to optimize cognitive function for individuals comprising the administration of a composition comprising one or more SNR1 or buproprion to an individual.

11. The method according to claim 10, wherein said individuals are selected from the group consisting of: individuals taking exams, servicemen and officers in the Armed Services during exercises or armed conflict, students, athletes during sporting events, and individuals in various work-settings.

12. The method according to claim 10, wherein said SNR1 composition is administered to the individual as needed, before, or during activities that require optimized cognitive function.

13. The method according to claim 1, wherein a medication or composition comprising both buproprion and one or more SNR1 are administered to an individual.

14. The method according to claim 4, wherein a medication or composition comprising both buproprion and one or more SNR1 are administered to an individual.

15. The method according to claim 8, wherein a medication or composition comprising both buproprion and one or more SNR1 are administered to an individual.

16. The method according to claim 10, wherein a medication or composition comprising both buproprion and one or more SNR1 are administered to an individual.

17. The method according to claim 1, wherein the amount of SNR1 or buproprion in said medication or composition is varied from day to day.

18. The method according to claim 13, wherein the amount of SNR1 and buproprion in said medication or composition is varied from day to day.

19. The method according to claim 4, wherein the amount of SNR1 or buproprion in said medication or composition is varied from day to day.

20. The method according to claim 14, wherein the amount of SNR1 and buproprion in said medication or composition is varied from day to day.

21. The method according to claim 8, wherein the amount of SNR1 or buproprion in said medication or composition is varied from day to day.

22. The method according to claim 15, wherein the amount of SNR1 and buproprion in said medication or composition is varied from day to day.

23. The method according to claim 10, wherein the amount of SNR1 or buproprion in said medication or composition is varied from day to day.

24. The method according to claim 16, wherein the amount of SNR1 and buproprion in said medication or composition is varied from day to day.
